(19) United States
(12) Patent Application Publication
(10) Pub. No.: US 2026/0207907 A1
(43) Pub. Date: Jul. 23, 2026

(54) MICRONEEDLE PATCH AND METHOD OF MANUFACTURING MICRONEEDLE PATCH

(71) Applicant: RAPHAS CO., LTD., Seoul (KR)

(72) Inventors: Seong-Jun KIM, Gimpo-si, Gyeonggi-do (KR); Yu-JI CHOI, Seoul (KR); Kwang-Su KIM, Yongin-si, Gyeonggi-do (KR); Do-Hyeon JEONG, Seoul (KR); Soo-Kie LA, Seoul (KR); Na-Young YOO, Gimpo-si, Gyeonggi-do (KR); Ju-Young JIN, Goyang-si, Gyeonggi-do (KR)

(73) Assignee: RAPHAS CO., LTD., Seoul (KR)

(21) Appl. No.: 19/138,690

(22) PCT Filed: Dec. 19, 2022
(86) PCT No.: PCT/KR2022/020734

§ 371 Date: Jun. 13, 2025

(51) Int. Cl. A61M 37/00

(52) U.S. Cl. CPC A61M 37/0015; A61M 2037/0023; A61M 2037/0053

(57) ABSTRACT

A microneedle patch of the present invention comprises an adhesive sheet configured to adhere to skin, a needle support disposed on the adhesive sheet, and a needle body disposed on the needle support and having a cutting edge, wherein at least one of the needle support and the needle body comprises a swellable polymeric material that is configured to expand upon contact with body fluid.

### TECHNICAL FIELD

The present invention relates to a microneedle patch and a method of manufacturing the microneedle patch.

### BACKGROUND ART

Although numerous drugs and bioactive substances have been developed for treatment of diseases, there are still issues that need to be improved in delivery of the drugs and the bioactive substances into the human body, such as a problem of crossing biological barriers (e.g., skin, oral mucous membrane, blood-brain barrier and the like) and efficiency of drug delivery.

The drugs and the bioactive substances are generally administered orally in a tablet or capsule form, but many drugs cannot be effectively delivered by the above methods of administration alone due to digestion or absorption in gastrointestinal tract or loss by hepatic mechanisms. In addition, some drugs cannot effectively diffuse across intestinal mucosa. In addition, patient compliance is also an issue (e.g., in case of patients who need to take medication at specific intervals, in case of seriously ill patients who are unable to take medication or the like).

Another common technique for the delivery of the drugs and bioactive substances is use of conventional needles. While this method is effective compared to oral administration, there are problems causing pain at an injection site, localized damage to skin, bleeding, and disease infection at the injection site.

To solve the above problems, various microneedle patches including microneedles have been developed. The microneedle patches developed so far have been mainly used for drug delivery within a living organism, blood collection, and analyte detection in the body.

Unlike the conventional needles, the microneedles are characterized by painless skin penetration and non-trauma. In this case, in order to penetrate the skin, the microneedle should have penetrability, and as the microneedle should penetrate stratum corneum of 10-20 μm, which is the strongest obstacle in the skin, the microneedle is required to have sufficient physical hardness. In addition, an appropriate length to reach capillaries for increasing the efficiency of drug delivery should also be considered.

Conventional microneedles have been limited to materials such as silicon, polymer, metal, glass, etc. due to limitations in manufacturing methods, and have disadvantages such as drug denaturation, insufficient hardness, and drug loss due to complicated and long manufacturing time by using molding technology. Therefore, there is a continuous need for microneedles that have a diameter small enough to realize painless skin penetration and a sufficient length to penetrate deeply into the skin, while achieving the sufficient hardness without special restrictions on materials and minimizing the drug loss.

### DISCLOSURE OF INVENTION

### Technical Problem

To solve the above problems, it is an object of the present invention to provide a microneedle patch that includes a microneedle formed of swellable polymeric material that expands upon contact with a body fluid and a method for manufacturing the same.

Further, it is an object of the present invention to provide a microneedle patch that has penetrability and sufficient hardness, and improves adhesive force so as to effectively introduce a drug and a method of manufacturing the same.

### Solution to Problem

To achieve the object, the present invention may provide a microneedle patch comprising: an adhesive sheet configured to adhere to skin; a needle support disposed on the adhesive sheet; and a needle body disposed on the needle support and having a cutting edge, wherein at least one of the needle support and the needle body comprises a swellable polymeric material that is configured to expand upon contact with body fluid.

Here, at least one of the needle support and the needle body may comprise a drug, and at least one of the needle support and the needle body may be configured to expand and deliver the drug, upon contact with the body fluid.

Further, at least one of the needle support and the needle body may be formed by solidifying a viscous composition in which the swellable polymeric material and a drug are dissolved.

Moreover, the needle body may comprise the swellable polymeric material and may be configured in a shape having a base diameter D corresponding to a diameter of a bottom surface abutting the needle support, a needle height H corresponding to an orthogonal distance from the bottom surface to a top end, and a needle diameter N corresponding to a diameter of the cutting edge, and at least one of the base diameter, the needle height, and the needle diameter of the needle body may increase upon contact with the body fluid.

Meanwhile, the base diameter and the needle diameter may increase by more than 1.1 times and less than 2 times, upon contact with the body fluid.

Moreover, the needle body may comprise the swellable polymeric material and may be configured in a shape having a base diameter D corresponding to a diameter of a bottom surface abutting the needle support, a needle height H corresponding to an orthogonal distance from the bottom surface to a top end, and a needle diameter N corresponding to a diameter of the cutting edge, and any one of the base diameter, the needle height, and the needle diameter of the needle body may increase and any one of others may decrease, upon contact with the body fluid.

In addition, the adhesive sheet and the needle support may form a single adhesive surface, and the needle support may be disposed by being inserted inwardly into the adhesive sheet from the adhesive surface and the needle body may be formed by extending outwardly from the adhesive surface.

Moreover, the needle supports may be disposed as a plurality of engraved patterns inserted inwardly into the adhesive sheet and spaced apart from one another, and the needle bodies may be disposed on the needle supports, respectively.

In addition, the needle support may be disposed as a layer inserted inwardly into the adhesive sheet and extending parallel, and the needle bodies may be disposed to be spaced apart from one another on the needle support.

Meanwhile, to achieve the object described above, the present invention may provide a method of manufacturing a microneedle patch, comprising: forming a needle support on a base resin; forming an adhesive sheet on the base resin to cover the needle support; forming a cover resin on an upper portion of the adhesive sheet and inverting the needle support and the adhesive sheet such that the base resin is disposed above the needle support and the adhesive sheet; removing the base resin; and forming a needle body on the needle support, wherein at least one of the needle support and the needle body comprises a swellable polymeric material that is configured to expand upon contact with body fluid.

Here, at least one of the needle support and the needle body may be formed by solidifying a viscous composition in which the swellable polymeric material and a drug are dissolved.

Further, the forming of the needle body may include: providing a pair of structures in each of which a base needle is formed on the needle support; relatively moving the pair of structures closer to each other so that a pair of base needles formed at the pair of structures respectively, contact each other; relatively moving the pair of structures away from each other so that the pair of base needles are elongated while adhering to each other and are deformed; and forming the needle body by the deformed base needle.

Meanwhile, the needle support may include a plurality of supports spaced apart and formed on the base resin, the adhesive sheet may be configured to cover each of the plurality of supports, and the needle body may be formed on each of the plurality of supports.

In addition, the needle bodies may be formed by being disposed to be spaced apart from one another on the needle support.

### Advantageous Effects of Invention

According to the microneedle patch and the method of manufacturing the same of the present invention that have the aforementioned configuration, there is an advantage that the microneedle patch expands to increase its surface area and volume upon contact with a body fluid, thereby enabling effective drug delivery.

### BRIEF DESCRIPTION OF DRAWINGS

FIG. 1 is a diagram schematically illustrating a process for fabricating a sheet of a microneedle patch according to one embodiment of the present invention,

FIG. 2 is a plan view photograph of the sheet fabricated according to FIG. 1,

FIG. 3 is a diagram schematically illustrating a process for fabricating the microneedle patch according to one embodiment of the present invention using the sheet according to FIG. 1,

FIG. 4 is a side view photograph of the microneedle patch fabricated according to the process of FIG. 3,

FIG. 5 is a diagram schematically illustrating a process for fabricating a sheet of a microneedle patch according to another embodiment of the present invention,

FIG. 6 is a plan view photograph of the sheet fabricated according to FIG. 5,

FIG. 7 is a diagram schematically illustrating a process for fabricating the microneedle patch according to another embodiment of the present invention using the sheet according to FIG. 5,

FIG. 8 is a side view photograph of the microneedle patch fabricated according to the process of FIG. 7,

FIG. 9 is a diagram schematically illustrating changes of a needle body in the microneedle patch according to one embodiment of the present invention,

FIGS. 10 through 13 are photographs of experimental changes in the needle body in the microneedle patch according to one embodiment of the present invention,

FIG. 14 is a diagram schematically illustrating changes of a needle body in the microneedle patch according to another embodiment of the present invention.

### BEST MODE FOR CARRYING OUT THE INVENTION

Hereinafter, with reference to the accompanying drawings, a microneedle patch according to an embodiment of the present invention and a method of manufacturing the same will be specifically described.

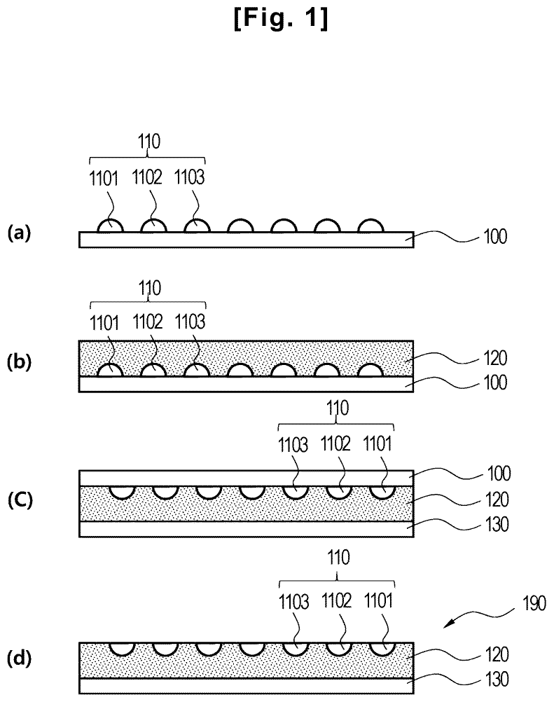

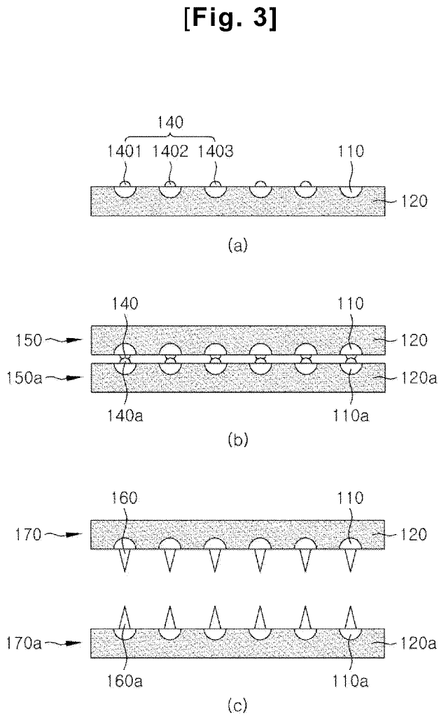

FIG. 1 is a diagram schematically illustrating a process for fabricating a sheet 190 of a microneedle patch according to one embodiment of the present invention, and FIG. 3 is a diagram schematically illustrating a process for fabricating the microneedle patch according to one embodiment of the present invention using the sheet 190 according to FIG. 1.

The microneedle patch according to one embodiment of the present invention may be formed through the processes of FIGS. 1 and 3. That is, it may be understood that FIGS. 1 and 3 are drawings illustrating the processes of manufacturing the microneedle patch of the present invention, and that a shape shown in (c) of FIG. 3, which corresponds to a final step, represents microneedle patches 170, 170a.

First, a needle support 110 may be formed by being spotted onto a first base resin 100, as shown in (a) of FIG. 1. The first base resin 100 corresponds to a temporarily prepared sheet for forming the needle support 110. That is, the first base resin 100 is omitted from the finalized sheet 190 for the patch.

The first base resin 100 may be configured in a shape of a flat plate and may be provided, for example, in a form of a synthetic resin and a transparent PET film.

The needle support 110 may be formed by spotting a plurality of needle supports on an upper portion or a top portion of the first base resin 100. For example, the needle support 110 may comprise the plurality of supports, including a first support 1101, a second support 1102, and a third support 1103 spaced apart from one another. Each of the supports 1101, 1102, 1103 may have the same shape as one another and may be disposed in sequence.

In this case, the needle support 110 may be formed by spotting and solidifying the first viscous compositions to be spaced apart from one another by a predetermined interval on a top surface of the first base resin 100. Accordingly, each of the supports 1101, 1102, 1103 may be formed in a slightly different shape under influence of adhesion, draft, or the like, although an overall shape is the same. The first viscous composition for forming the needle support 110 will be specifically described later.

Further, the needle support 110 may be formed in various shapes on the first base resin 100 through various methods. Moreover, each support 1101, 1102, 1103 may form a roughly circular, oval, or curved surface by surface tension, and may be formed in a hemispherical shape (hereinafter, referred to as “hemispherical”) on the first base resin 100.

In this case, the arrangement, spacing, or number of each support 1101, 1102, 1103 may be formed differently as required. For example, each support 1101, 1102, 1103 may form a grid and may be regularly arranged.

Meanwhile, as shown in (b) of FIG. 1, an adhesive sheet 120 may be formed on an upper portion or a top portion of the first base resin 100 and the needle support 110. That is, the adhesive sheet 120 may be formed on the upper (or top) portion of the first base resin 100 to cover the needle support 110. Accordingly, the needle support 110 is disposed between the adhesive sheet 120 and the first base resin 100.

The adhesive sheet 120 may be formed above the needle support 110 to completely cover the needle support 110.

Then, the first cover resin 130 is provided by being attached to an upper portion of the adhesive sheet 120 in the state of (b) of FIG. 1. Thereafter, the needle support 110 and the adhesive sheet 120 are inverted or turned upside down as shown in (c) of FIG. 1 such that the first base resin 100 is disposed on an upper side. That is, a shape shown in (b) of FIG. 1 is flipped or reversed so that the first cover resin 130 is disposed on a bottom side and the first base resin 100 is disposed on the upper side.

In this case, the first cover resin 130 corresponds to a bottom surface and the first base resin 100 corresponds to a top surface. The first cover resin 130, like the first base resin 100, may be provided in the shape of the flat plate and may be provided, for example, in the form of the synthetic resin and a transparent PET film. In other words, the first cover resin 130 and the first base resin 100 may be provided in the same configuration or in different configurations, and may be designated by different terms for convenience of description.

As shown in (d) of FIG. 1, the first base resin 100 is removed, and the needle support 110 and the adhesive sheet 120 are provided in a predetermined shape on an upper portion or a top portion of the first cover resin 130 to produce the sheet 190 of the microneedle patch.

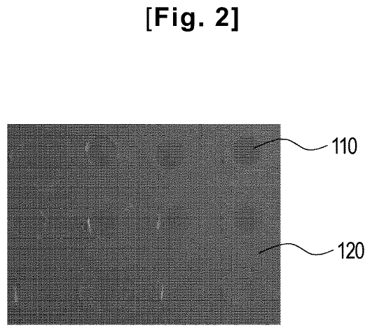

FIG. 2 is a plan view photograph of the sheet 190 fabricated according to the process of FIG. 1.

Referring to FIG. 1 and FIG. 2, the needle support 110 is disposed with being inserted into an interior of the adhesive sheet 120. For example, it may be appreciated that the needle support 110 is disposed by being inserted into the adhesive sheet 120 in an engraved pattern that is recessed inwardly. In this case, the engraved pattern may be formed, for example, in an approximately hemispherical, curved or semi-elliptical shape.

Here, the needle support 110 having the hemispherical shape is described by way of example, and the needle support 110 may be formed in various shapes other than hemispherical, such as curved, semi-elliptical, and the like.

Further, the needle support 110 may be inserted into the adhesive sheet 120 to form one side of the adhesive sheet 120. As described above, since the first base resin 100 is configured to be flat, the needle support 110 and the adhesive sheet 120 which are formed by adhering thereto may form a flat single surface. Then, when the first base resin 100 is removed, the single surface formed by the needle support 110 and the adhesive sheet 120 is exposed. Hereinafter, such a single surface is referred to as an adhesive surface. For example, the adhesive surface may refer to a surface that contacts the skin.

Further, as described above, each of the supports 1101, 1102, 1103 is disposed spaced apart from each other, and between the supports 1101, 1102, 1103, the adhesive sheet 120 is exposed and disposed. Accordingly, when the adhesive surface is in contact with the skin, adhesion may be increased through the adhesive sheet 120 disposed between the supports 1101, 1102, 1103. Moreover, each support 1101, 1102, 1103 may be adhered more effectively due to the exposed adhesive sheet, which may increase administration efficiency.

FIG. 3 is a diagram schematically illustrating a process for fabricating microneedle patches 170, 170a according to one embodiment of the present invention using the sheet according to FIG. 1. The following will be discussed in order.

First, as shown in (a) of FIG. 3, a second viscous composition for forming a base needle 140 may be spotted on an upper portion or a top portion of the needle support 110. The second viscous composition may be composed of the same material as the first viscous composition for the needle support 110 described above or the different materials. This will be further discussed later.

The base needle 140 may be formed in the number corresponding to the number of the needle support 110. For example, a plurality of base needles 140 may be formed, including a first base 1401, a second base 1402, and a third base 1403 formed on the supports 1101, 1102, and 1103, respectively. More specifically, the first base 1401 is formed on an upper portion of the first support 1101, the second base 1402 is formed on an upper portion of the second support 1102, and the third base 1403 is formed on an upper portion of the third support 1103.

Although not shown in the accompanying drawings, it is also possible for a plurality of base needles 140 to be spotted on the upper surface of the single needle support 110. In this case, the base needles 140 may be spaced apart and spotted on an upper surface or a top surface of the single needle support 110.

Meanwhile, each of the supports 1101, 1102, 1103 or each of the bases 1401, 1402, 1403 is referred to separately for convenience of description, and each of them corresponds to the same configuration. In addition, each of the supports 1101, 1102, 1103 and each of the bases 1401, 1402, 1403 may be formed by the viscous composition and may be formed in a slightly different shape, but this is not essential to the nature of the present invention.

In summary, the base needle(s) 140 may be spotted on the upper surface of the plurality of supports 1101, 1102, 1103 spaced apart from one another, and the base needle 140 may be formed by projecting upwardly from the adhesive surface which is formed by the needle support 110 and the adhesive sheet 120. Hereinafter, a shape as shown in (a) of FIG. 3 is referred to as a structure 150.

Then, as shown in (b) of FIG. 3, a pair of structures 150, 150a are disposed to face each other. At this time, each of the structural body 150, 150a is formed the same as in (a) of FIG. 3 described above, and corresponds to the same configuration as each other.

Thus, a first structure 150 includes a first needle support 110, a first adhesive sheet 120, and a first base needle 140, and a second structure 150a includes a second needle support 110a, a second adhesive sheet 120a, and a second base needle 140a.

Particularly, the first structure 150 and the second structure 150a are arranged so that the first base needle 140 and the second base needle 140a face each other. That is, an adhesive surface of the first structure 150 and an adhesive surface of the second structure 150a are arranged to face each other. Then, the first structure 150 and the second structure 150a are moved relative to each other, so that the first base needle 140 and the second base needle 140a are in contact with each other.

For example, the second structure 150a is positioned so that the adhesive surface faces upward. Then, the first structure 150 is positioned on a top of the second structure 150a so that the adhesive surface thereof is oriented toward the bottom surface. Then, the first structure 150 is lowered so that the first base needle 140 and the second base needle 140a are in contact with each other.

At this time, the first needle support 110, the second needle support 110a, the first adhesive sheet 120 and the second adhesive sheet 120a are arranged so as not to contact one another. Further, the needle supports 110, 110a and the adhesive sheets 120, 120a are completely solidified and do not deform even when they are in contact with one another.

In contrast, the first base needle 140 and the second base needle 140a may be deformed by contacting each other since their viscous compositions are not solidified. That is, the structures 150, 150a are placed in contact with each other while the base needles 140, 140a are not fully solidified.

Subsequently, as shown in (c) of FIG. 3, the structures 150, 150a are moved relative to each other in a direction away from each other. Thereby, the base needles 140, 140a are stretched by each other and then solidified.

The base needles 140, 140a adhered to each other form needle bodies 160, 160a after tensile and solidification steps. Accordingly, microneedle patches 170, 170a of the present invention may be manufactured. That is, the microneedle patches 170, 170a include the needle supports 110, 110a, the adhesive sheets 120, 120a, and the needle bodies 160, 160a.

The process by which the needle bodies 160, 160a are formed will be more specifically described below.

As described above, the first base needle 140 and the second base needle 140a are arranged with adhering to each other. Then, the first needle support 110 and the first adhesive sheet 120 are moved relatively in a direction away from the second needle support 110a and the second adhesive sheet 120a.

Accordingly, when the adhered base needles 140, 140a are moved away, ends thereof are extended by the adhesive force and a radius of each base needle becomes smaller. Eventually, the needle bodies 160, 160a that are separated into pairs and have a cutting edge, may be formed respectively.

For example, as shown in FIG. 3, an approximately hemispherical base needles 140, 140a is transformed into an approximately cone-shaped needle bodies 160, 160a by stretching the adhered ends thereof.

That is, the base needles 140, 140a adhered to each other may be tensioned or elongated to a desired length at a predetermined speed (or velocity) to form the needle bodies 160, 160a. For example, the base needles 140, 140a that are adhered to each other are 1) firstly stretched or elongated by a first length at a first speed (first stretching or elongating), 2) waited for a predetermined amount of time while the stretching or the elongation is stopped (waiting or standing by), 3) secondly stretched or elongated by a second length at a second speed (second stretching or elongating), and 4) separated from each other (cutting). In a separation or cutting step, the base needles 140, 140a may be cut by rapidly moving them in a fully solidified state, or may be cut using a cutting device such as a laser.

For example, the base needles 140, 140a that are adhered to each other are stretched by 10 to 5000 μm at 0.8 to 600,000 μm/s without blowing, are waited for (or stand by) 1 to 100 seconds after stopping the stretching, and then are secondly stretched by 10 to 5000 μm at 0.8 to 600,000 μm/s during blowing to the viscous composition at a wind speed of 1 to 100 m/s, and then are cut by solidifying the viscous composition with blowing at a wind speed of 5 to 100 m/s.

At this time, the first speed, the first length, the waiting time, the second speed, the second length, and the blowing or not blowing may be set differently as required, which may be determined according to the required result. In particular, the longer the waiting time with stopping the stretching after the first stretching is increased, the larger a diameter of a middle portion thereof may be, so that the strength may be relatively greater. The method mentioned above is described in detail in Korean Patent No. 10-1254240 of the applicant.

In summary, the microneedle patches 170, 170a are formed as follows by: 1) forming the needle support 110 on the upper or top portion of the first base resin 100; 2) forming the adhesive sheet 120 on the upper or top portion of the first base resin 100 to cover the needle support 110; 3) forming the first cover resin 130 on the upper or top surface of the adhesive sheet 120 and disposing the elements 110-130 upside down; 4) removing the first base resin 100 to expose the adhesive surface; 5) providing a pair of structures 150, 150a in each of which the base needle 140 is formed on the adhesive surface to correspond to a position of the needle support 110; 6) relatively moving the pair of structures 150, 150a closer to each other so that base needles 140, 140a are adhered to each other; and 7) relatively moving the pair of structures 150, 150a further away from each other so that the adhered base needles 140, 140a are pulled or elongated together and form the needle bodies 160, 160a.

Meanwhile, in the embodiment of FIG. 3 described above, it is shown, but not limited to, that both the upper and lower structures 150, 150a are formed by spotting bass needles 140, 140a.

For example, although not shown in the drawings, the second bass needle 140a may be spotted only on the lower second structure 150a. That is, no base needle may be spotted on the first needle support 110 of the upper first structure 150. In this case, when the first structure 150 and the second structure 150a are relatively moved in the direction closer to each other, a surface of the first needle support 110 of the first structure 150 may be moved to contact the second base needle 140a of the second structure 150a. After the surface of the first needle support 110 of the first structure 150 contacts the second base needle 140a of the second structure 150a, the method of stretching the second base needle 140a by relative movement of the first structure 150 and the second structure 150a in a direction away from each other is similar to the above-mentioned embodiment, and therefore will not be repeatedly described.

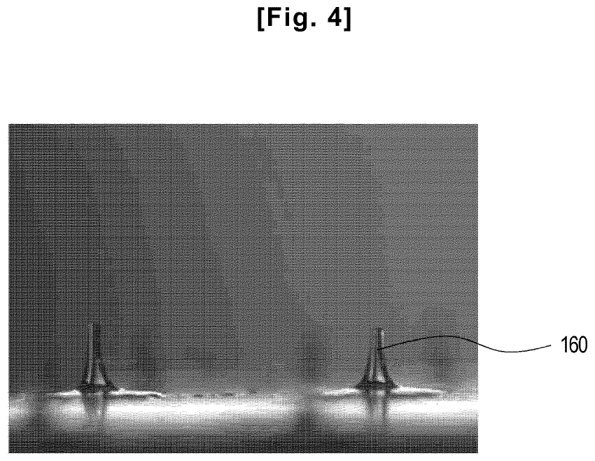

FIG. 4 is a side view photograph of the microneedle patch fabricated according to the process of FIG. 3.

As shown in FIG. 4, it may be noted that the needle body 160 has been formed. Meanwhile, in FIG. 4, the needle body 160 is formed by a viscous composition containing Chitosan as swellable polymeric material or swellable hydrophilic polymers described later. Such swellable polymeric material or swellable hydrophilic polymers will be specifically described later.

Meanwhile, the above-described shapes of the microneedle patches 170, 170a are exemplary, and the microneedles of the present invention may be manufactured in various shapes. Hereinafter, microneedles having different shapes of needle structures will be described. However, microneedle patches 270, 270a as below may be manufactured using the same process as the previously described with regard to the microneedle patches 170, 170a. Therefore, for the same process, the above description will be applied and detailed description will be omitted, and the corresponding configurations will use the same terms and will be distinguished by drawing symbols.

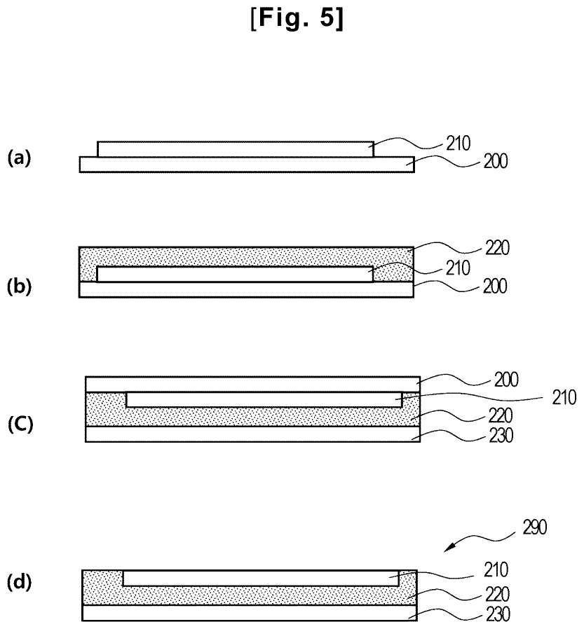

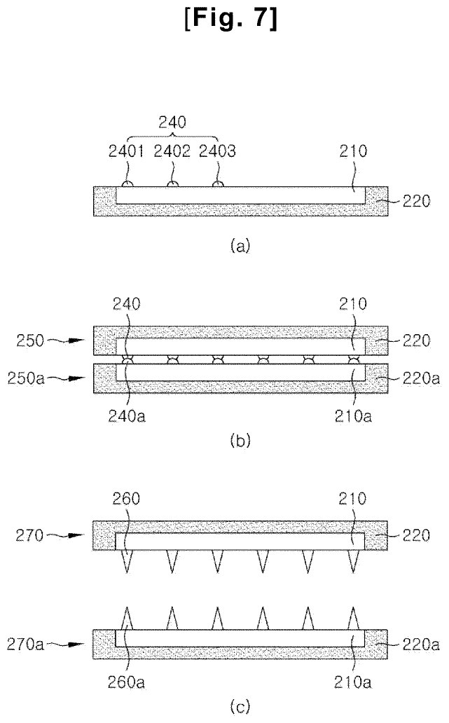

FIG. 5 is a diagram schematically illustrating a process for forming a sheet 290 of a microneedle patch according to another embodiment of the present invention, and FIG. 7 is a diagram schematically illustrating a process for fabricating the microneedle patches 270, 270a according to another embodiment of the present invention using the sheet 290 according to FIG. 5.

The microneedle patches 270, 270a according to another embodiment of the present invention may be formed through the process of FIGS. 5 and 7. That is, it may be appreciated that FIGS. 5 and 7 are drawings illustrating the process for fabricating microneedle patches 270, 270a of the present invention, and that a shape shown in (c) of FIG. 7, which corresponds to a final step, represents the microneedle patches 270, 270a.

As shown in (a) of FIG. 5, a third needle support 210 may be formed on a second base resin 200. The third needle support 210 may be formed by extending along an upper portion or a top portion of the second base resin 200. In this case, the third needle support 210 may be formed by coating the first viscous composition described above on the upper or top portion of the second base resin 200.

Subsequently, as shown in (b) of FIG. 5, an adhesive sheet 220 may be formed on the upper or top portions of the second base resin 200 and the third needle support 210. That is, the adhesive sheet 220 may be formed on the upper or top portion of the second base resin 200 to cover the third needle support 210. Accordingly, the third needle support 210 is disposed between the adhesive sheet 220 and the second base resin 200.

Then, in a state of (b) of FIG. 5, a second cover resin 230 is provided on an upper portion or a top portion of the adhesive sheet 220, and then, as shown in (c) of FIG. 5, these components 200-230 are flipped over so that the second base resin 200 is arranged on the upper portion. That is, upper and lower sides of the third needle support 210 and the adhesive sheet 220 are swapped or reversed.

Subsequently, as shown in (d) of FIG. 5, the second base resin 200 on the upper or top portion is removed, and the third needle support 210 and the adhesive sheet 220 are configured with a predetermined shape.

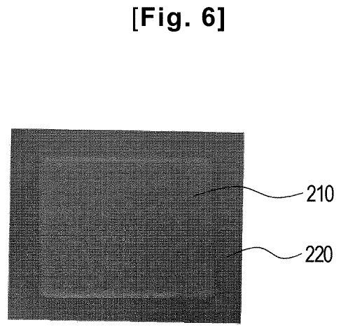

FIG. 6 is a plan view photograph of the sheet 290 fabricated according to FIG. 5.

Referring to FIG. 5 and FIG. 6, the third needle support 210 is inserted into an interior of the adhesive sheet 220 and is disposed in a form of a single layer, i.e., in a form of a layer extending in parallel. In other words, the third needle support 210 is provided in a state of being inserted entirely inwardly into the adhesive sheet 220. For example, the third needle support 210 may be recessed or embedded into the adhesive sheet 220 in a shape of a flat plate. In this case, the adhesive sheet 220 may be exposed along an edge of the third needle support 210.

Further, the third needle support 210 may be inserted into the adhesive sheet 220 to form one side of the adhesive sheet 220. As described above, since the second base resin 200 is configured to be flat, the third needle support 210 and the adhesive sheet 220 which are adhered thereto may form a flat single surface. Hereinafter, such a single surface is referred to as an adhesive surface. For example, the adhesive surface means a surface abutting an affected area.

FIG. 7 is a diagram schematically illustrating the process for fabricating the microneedle patches 270, 270a according to another embodiment of the present invention using the sheet 290 according to FIG. 5. The following will be discussed in order.

First, as shown in (a) of FIG. 7, a second viscous composition for forming a base needle 240 may be spotted on an upper portion or a top portion of the third needle support 210. The second viscous composition may be composed of the same materials as the first viscous composition for the third needle support 210 or the different materials. This will be further discussed later.

A plurality of base needles 240 may be formed on the third needle support 210. For example, the bass needle 240 may include a first base 2401, a second base 2402, and a third base 2403 formed spaced apart from one another. Each of the bases 2401, 2402, 2403 may be formed in slightly different shapes, but this is not essential to the nature of the present invention.

Unlike the needle support 110 described above, the third needle support 210 is formed by extending in a form of a flat sheet. Accordingly, a plurality of base needles 240 may be formed on the single third needle support 210. Such a shape has an advantage that it is more convenient to manufacture and may more effectively support the needle bodies 260, 260a which will be described later.

In summary, a plurality of base needles 240 may be spaced apart from one another and may be spotted on the upper or top surface of the third needle support 210, and the base needles 240 may be formed by protruding upwardly on the adhesive surface which is formed by the third needle support 210 and the adhesive sheet 220. Hereinafter, a shape as shown in (a) of FIG. 7 is designated as a structure 250.

Then, as shown in (b) of FIG. 7, a pair of structures 250, 250a are disposed to face each other. In this case, each of the structures 250, 250a is formed to be the same as in (a) of FIG. 7 described above and corresponds to the same configuration as each other.

Thus, the third structure 250 includes a third needle support 210, a third adhesive sheet 220, and a third base needle 240, and the fourth structure 250a includes a fourth needle support 210a, a fourth adhesive sheet 220a, and a fourth base needle 240a.

Specifically, the third structure 250 and the fourth structure 250a are disposed so that the third base needle 240 and the fourth base needle 240a face each other. That is, an adhesive surface of the third structure 250 and an adhesive surface of the fourth structure 250a are disposed to face each other. Then, the third structure 250 and the fourth structure 250a are moved relative to each other, so that the third base needle 240 and the fourth base needle 240a are in contact with each other.

For example, the fourth structure 250a is positioned so that the adhesive surface faces upward. Then, the third structure 250 is placed on an upper portion or top portion of the fourth structure 250a such that the adhesive surface thereof faces the bottom surface. Then, the third structure 250 is lowered so that the third base needle 240 and the fourth base needle 240a are in contact with each other.

At this time, the third needle support 210 and the fourth needle support 210a are disposed so as not to contact each other, and the third adhesive sheet 220 and the fourth adhesive sheet 220a are disposed so as not to contact each other. Further, each of needle support 210, 210a and each of adhesive sheet 220, 220a are completely solidified and do not deform even when they are in contact with one another.

In contrast, the third base needle 240 and the fourth base needle 240a are subject to shape deformation by contacting each other because their viscous compositions are not solidified. That is, the structures 250, 250a are disposed to be in contact with each other while the base needles 240, 240a are not fully solidified.

Subsequently, as shown in (c) of FIG. 7, the structures 250, 250a are moved relative to each other in a direction away from each other. Thereby, the base needles 240, 240a are stretched or elongated by each other and then solidified.

The base needles 240, 240a adhered to each other are formed into needle bodies 260, 260a through tensile and solidification steps. Accordingly, the microneedle patch 270, 270a of the present invention may be manufactured. That is, the microneedle patch 270, 270a includes the needle supports 210, 210a, the adhesive sheets 220, 220a, and the needle bodies 260, 260a.

The process by which the needle bodies 260, 260a are formed has been described above in the preceding embodiments, and therefore, a repetitive description is omitted.

As such, the microneedle patches 270, 270a may form the needle supports 210, 210a of a different shape than the needle supports in the microneedle patches 170, 170a described above. Further, the microneedle patches 270, 270a may form the needle bodies 260, 260a of the same shape as the needle bodies in the microneedle patches 170, 170a described above. Hereinafter, the needle bodies 160, 160a, 260, 260a will be described in detail.

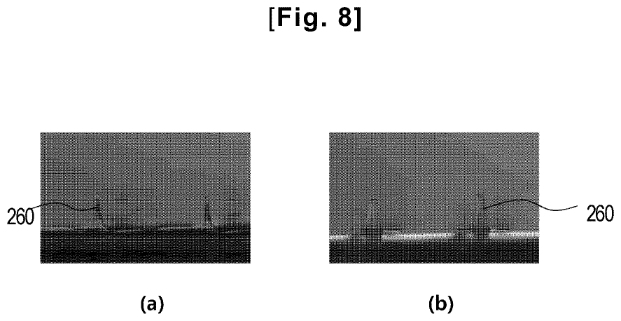

FIG. 8 is a side view photograph of the microneedle patch fabricated according to the process of FIG. 7.

As shown in FIG. 8, it may be observed that the needle body 260 is formed. Meanwhile, in (a) of FIG. 8, the needle body 260 is formed by a viscous composition containing Chitosan as the swellable polymeric material or swellable hydrophilic polymer to be described later, and in (b) of FIG. 8, the needle body 260 is formed by a viscous composition containing Polyvinyl Alcohol (PVA) as the swellable polymeric material or swellable hydrophilic polymer to be described later. Such swellable polymeric material or swellable hydrophilic polymer will be specifically described later.

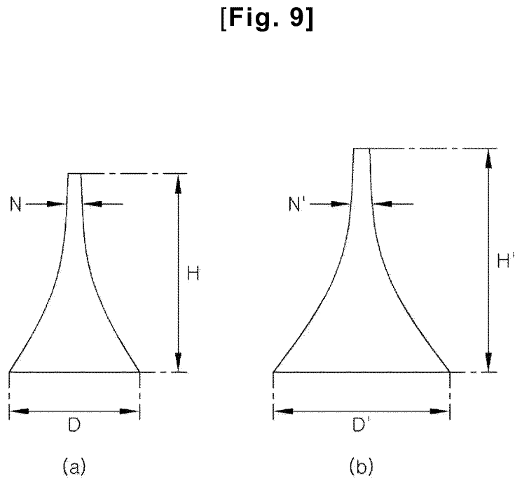

FIG. 9 is a diagram schematically illustrating changes in the needle body of the microneedle patch according to one embodiment of the present invention. (a) and (b) of FIG. 9 illustrate only the needle bodies 160, 160a, 260, 260a in the above-described microneedle patches 170, 170a, 270, 270a. Hereinafter, the single needle body 160 will be described in detail and such a description will be incorporated by reference.

Referring to FIG. 9, the needle body 160 may be configured in a shape of an approximately circular cone. That is, the needle body 160 may be configured in a shape of being formed in an approximately circular shape at a base or a bottom and gradually narrowing in a diameter toward a top to form a cutting edge, an apex, or a peak. Hereinafter, a diameter of a bottom surface of the needle body 160 is referred to as a base diameter D, and the orthogonal distance from the bottom surface to the top end is referred to as a needle height H. In this case, the base diameter D may be appreciated as a cross-sectional diameter of a portion in contact with the needle support 110, and the needle height H may be appreciated as an orthogonal distance from the needle support 110 or the adhesive surface.

In this case, as described above, the needle body 160 corresponds to a structure formed by the solidification of the viscous composition. Accordingly, the bottom surface of the needle body 160 may not form a complete circle, and the base diameter D may be obtained as an approximate value. The needle height H may be formed to a set value as required, but may have a predetermined error in the separation or cutting step.

Meanwhile, the needle body 160 corresponds to a shape formed by cutting under a tensile force. Accordingly, the upper or top end forms a predetermined tensile fracture surface rather than a point, and a generatrix is formed as a curve. In this case, a curvature of the generatrix may be determined by a tensile or elongating speed or the like in the process of forming the needle body 160 as described above.

As described above, the needle body 160 may be formed by stretching or elongating at the first speed (or velocity) and then further stretching or elongating at the second speed (or velocity). In this case, the second speed may be faster than the first speed. Accordingly, as shown in FIG. 9, the needle body 160 may form a thinner and longer cutting edge (i.e., apex or peak) toward the top. In this case, a diameter of a portion stretched or elongated at the second speed is referred to as a needle diameter N. More specifically, the needle diameter N corresponds to the diameter of the cutting edge (i.e., apex or peak) of the needle body 160, and may be calculated as an average or median value thereof.

Furthermore, the top end of the needle body 160 may not form a complete shape up to a predetermined portion because such a top end forms a fracture surface. Accordingly, the top end diameter of the needle body 160 may be replaced by the needle diameter N.

Meanwhile, at least one of the needle body 160 and the needle support 110 as described above may contain a drug or medicine to be administered to the human body. That is, when the microneedle patch 170 according to the present invention is attached to the human body, the drug contained in at least one of the needle body 160 and the needle support 110 may be introduced into and delivered to the human body.

To this end, at least one of the needle body 160 and the needle support 110 may comprise the swellable hydrophilic polymer that swells upon contact with a body fluid.

When the microneedle patch 170 according to the present invention is attached to the human body and the needle body 160 or the needle support 110 swells upon contact with the body fluids, the drug contained inside the needle body 160 or the needle support 110 may be delivered into the human body. In this case, the needle body 160 or the needle support 110 may retain its shape or form without disintegrating or decomposing after contacting the body fluid. That is, the needle body 160 or the needle support 110 may retain its basic shape or form even when it expands.

Specifically, at least one of the needle body 160 and the needle support 110 may comprise the swellable polymeric material or swellable hydrophilic polymer, such as Polyvinyl Alcohol (PVA) or Chitosan. PVA and Chitosan are described by way of example of the swellable polymeric materials, and the swellable polymeric materials or swellable hydrophilic polymers are not limited thereto.

Consequently, at least one of the first viscous composition for the needle support 110 and the second viscous composition for the needle body 160 may comprise the drug and the swellable polymeric material. In this case, the first viscous composition and the second viscous composition may comprise the same swellable polymeric material, or may comprise different swellable polymeric materials.

Hereinafter, the viscous composition containing the drug that forms the needle body 160 or the needle support 110 will be described.

First, a case where distilled water (Di water) is used as a solvent and PVA is used as a polymeric material will be described. 1) The distilled water (Di water 50 to 80 wt % (weight percent)) is added to the stirrer or agitator and is heated up to 50 to 100° C. 2) PVA (5 to 30 wt %) is added and stirred at a speed of 200 to 800 RPM until it is completely dissolved. 3) After complete dissolution, a water-soluble polymer, such as Hydroxypropyl Methylcellulose (HPMC) (2 to 20 wt %) or Carboxymethyl Cellulose (CMC) (2 to 20 wt %), is added, and then is heated to 50 to 100° C. and stirred at a speed of 200 to 800 RPM. 4) Then, while maintaining 50 to 100° C., the drug (API: Active Pharmaceutical Ingredient), for example, Donepezil (5 to 30 wt %), is added and stirred at a speed of 200 to 800 RPM. 5) After complete dissolution, Glycerin (0.3 to 10 wt. %) is added at 30 to 80° C. and is stirred at a speed of 100 to 600 RPM until complete dissolution.

Then, a case where an aqueous solution of Acetic Acid is used as a solvent and Chitosan is used as a polymeric material is described. 1) The aqueous solution of Acetic Acid (50 to 80 wt. %) is added to the stirrer or agitator and is heated up to 30 to 80° C. 2) Chitosan (5 to 30 wt. %) is added and stirred at a speed of 200 to 800 RPM until it is completely dissolved. 3) After complete dissolution, the water-soluble polymer, for example, Hydroxypropyl Methylcellulose (HPMC) (2 to 20 wt. %) or Carboxymethyl Cellulose (CMC) (2 to 20 wt. %) is added, and is then heated to 30 to 80° C. and stirred at a speed of 200 to 800 RPM. 4) Then, while maintaining 50 to 100° C., the drug (API: Active Pharmaceutical Ingredient), for example, Donepezil (5 to 30 wt %), is added and stirred at a speed of 200 to 800 RPM. 5) After complete dissolution, Glycerin (0.3 to 10 wt. %) is added at 30 to 80° C. and stirred at a speed of 100 to 600 RPM until complete dissolution.

Donepezil corresponding to the drug described above is only a drug used to test the microneedle patch 170 according to the present invention, and the present invention is not limited to the above drug. For example, the types of drugs that may be contained in the needle body 160 or the needle support 110 are not particularly limited.

Meanwhile, the needle support 110 or the base needle 140 may be formed using the viscous composition which is prepared as described above, and the needle body 160 may be formed by deforming the needle support 110 or the base needle 140. At least one of the needle support 110 or the needle body 160 formed in this manner has the property of expanding or swelling in association with the body fluid.

For example, the needle support 110 and the needle body 160 may be formed from the same viscous composition or from the different viscous compositions. Here, the same viscous composition may be defined as having the same swellable polymeric material included in the composition and, further, all containing the drug.

Further, the different viscous compositions may be defined as only one of the compositions for the needle support 110 and the needle body 160 includes the swellable polymeric material, or the swellable polymeric materials included in the compositions are different, or whether the drug is contained in the composition is different.

For example, the needle support 110 may comprise PVA, and the needle body 160 may comprise chitosan, and vice versa. Further, at least one of the needle support 110 and the needle body 160 may comprise the drug.

Hereinafter, a case in which the needle body 160 comprises the swellable polymeric material will be described in detail. (a) of FIG. 9 illustrates an original state of the needle body 160, and (b) of FIG. 9 illustrates a state in which the needle body 160 has been expanded by contacting the body fluid.

The needle body 160 may change in the base diameter D, the needle height H, and the needle diameter N in response to engagement with the body fluid. In particular, at least one of the base diameter D, the needle height H, and the needle diameter N may be increased in a range from 1 to 200%. As a result, a surface area and volume of the needle body 160 may be increased.

For example, the needle body 160 may be formed with the base diameter D of approximately 807 μm, the needle height H of approximately 413 μm, and the needle diameter N of approximately 90 μm. Then, in the case of binding with the body fluid, the needle body 160 has a base diameter D′ of approximately 1048 μm, a needle height H′ of approximately 426 μm, and a needle diameter N′ of approximately 158 μm. Accordingly, the base diameter D has increased by about 30%, and the needle diameter N has increased by about 76%. Although the needle length H has changed little, it may be noted that the volume and surface area of the needle body 160 has increased as a result.

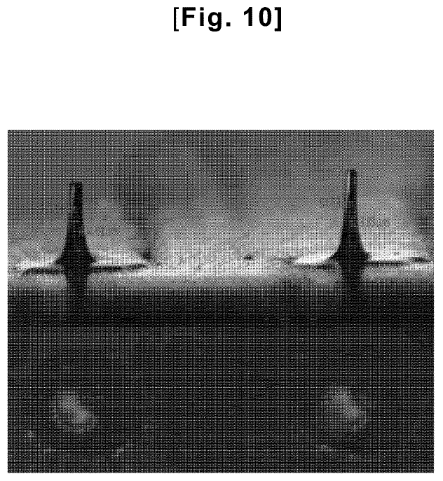

FIG. 10 to FIG. 13 are photographs of experimental changes in the needle body of the microneedle patch according to one embodiment of the present invention.

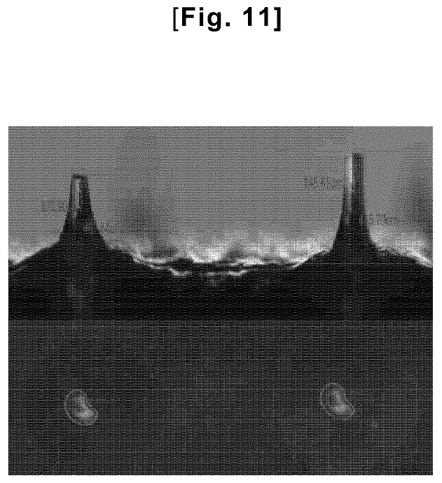

FIG. 10 and FIG. 11 correspond to the case where the needle body 160 comprises Chitosan among the swellable polymeric materials. FIG. 10 shows an original state of the needle body 160, and FIG. 11 shows a state after contacting a body fluid simulating solution (Phosphate Buffer Saline (PBS)).

For the needle bodies shown on the left in FIG. 10 and FIG. 11, the base diameter has been changed from 809.15 μm to 1029.4 μm, the needle height H has been changed from 404.91 μm to 407.84 μm, and the needle diameter N has been changed from 96.13 μm to 171.90 μm. Further, for the needle bodies shown on the right in FIG. 10 and FIG. 11, the base diameter has been changed from 806.33 μm to 1068.94 μm, the needle height H has been changed from 423.85 μm to 445.7 μm, and the needle diameter N has been changed from 84.53 μm to 145.65 μm.

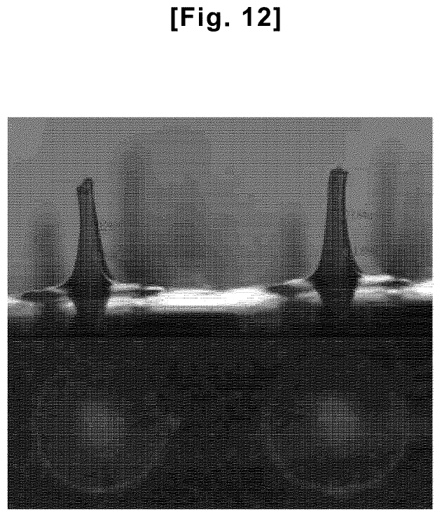

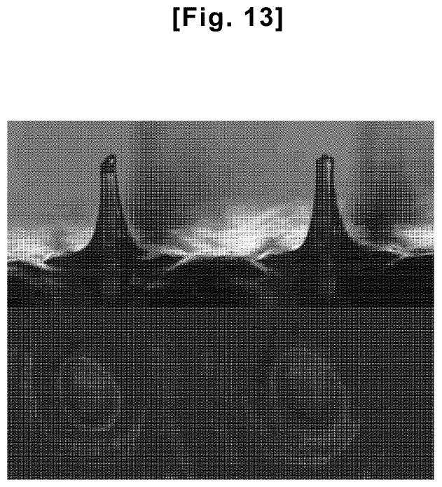

FIG. 12 and FIG. 13 correspond to a case in which the needle body 160 comprises PVA among the swellable polymeric materials. FIG. 12 shows an original state of the needle body 160, and FIG. 13 shows a state after contacting the body fluid simulating solution (phosphate buffer saline (PBS)).

For the needle bodies shown on the left in FIG. 12 and FIG. 13, the base diameter has been changed from 873.61 μm to 1002.10 μm, the needle height H has been changed from 614.74 μm to 614.67 μm, and the needle diameter N has been changed from 123.8 μm to 155.85 μm. Further, for the needle bodies shown on the right in FIG. 12 and FIG. 13, the base diameter has been changed from 888.55 μm to 1044.70 μm, the needle height H has been changed from 611.85 μm to 607.46 μm, and the needle diameter N has been changed from 123.84 μm to 167.5 μm.

From these experimental values, it may be observed that the surface area and volume of the needle body increase when the needle body is in contact with the body fluid. In this case, the needle height H remains somewhat unchanged, but the base diameter D and the needle diameter N increase, and thus it may be confirmed that the needle body expands in a lateral direction. To summarize, the base diameter D and the needle diameter N increase by 1.1 times or greater and less than 2 times when in contact with the body fluid, and the needle length H changes relatively little.

Further, at least one of the needle support 110 and the needle body 160 may comprise the drug as described above. That is, in the microneedle patch 170 of the present invention, 1) the needle body 160 may comprise the drug, 2) the needle support 110 may comprise the drug, or 3) both the needle body 160 and the needle support 110 may comprise the drug.

Table 1 attached below, lists experimental values measuring actual drug contents when the needle support or needle body includes the drug.

TABLE 1

| | A | B | C | D | E | F |
|---|---|---|---|---|---|---|
| Measured | 3.88 mg | 3.97 mg | 3.95 mg | 3.96 mg | 4.68 mg | 4.68 mg |
| Value | | | | | | |

In cases of A and B, 4 mg of drug was included only in the viscous composition to form the needle support, and the amount of drug was measured again after the needle support was actually formed. Then, A contained chitosan and B contained PVA. The results showed that 3.88 mg of drug was measured in case A, which was 97.0% of the theoretical value, and 3.97 mg of drug was measured in case B, which was 99.2% of the theoretical value. In other words, it may be observed that the drug is effectively contained even when only the needle support contains the drug.

In cases of C and D, 4 mg of drug was included only in the viscous composition for forming the needle body, and the amount of drug was measured again after actually forming the needle body. C contained Chitosan and D contained PVA. The results showed that 3.95 mg of drug was measured in case C, which was 98.7% of the theoretical value, and 3.96 mg of drug was measured in case D, which was 99.0% of the theoretical value. In other words, it may be observed that the drug is effectively contained even when only the needle body contains the drug.

In cases of E and F, 4.75 mg of drug were included in the viscous composition for forming the needle support and the needle body, and the amount of drug was measured again after actually forming the needle support and the needle body. E contained Chitosan and F contained PVA. The results showed that 4.68 mg of drug was measured in case E, which was 98.5% of the theoretical value, and 4.68 mg of drug was measured in case F, which was 98.6% of the theoretical value. In other words, it may be observed that the drug is effectively contained when the drug is included in both the needle support and the needle body.

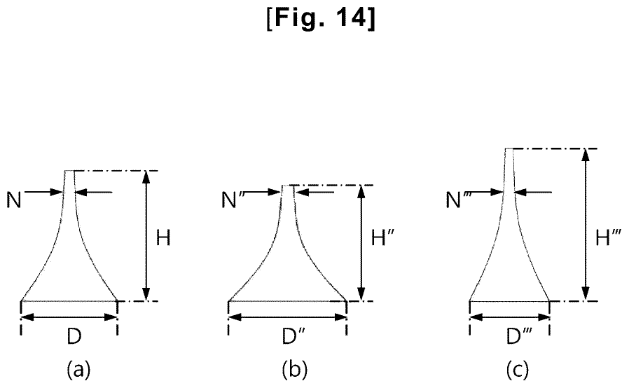

Meanwhile, FIG. 14 is a diagram schematically illustrating changes of the needle body in the microneedle patch according to another embodiment of the present invention.

Referring to FIG. 14, experiments of the applicant have shown that the needle body may be deformed such that any one of the base diameter D, the needle height H, and the needle diameter N increases and any one of the others, i.e., any one of remaining dimensions which are not increased, decreases, upon contact with the body fluid.

For example, a shape of (a) of FIG. 14 may be changed as shown in (b) of FIG. 14 upon contact with the body fluid. It may be seen that the needle body shown in (b) of FIG. 14 has a relatively increased base diameter D″, but a decreased needle height H″, compared to the shape in (a) of FIG. 14. In this case, the needle diameter N″ may be approximately the same or larger.

Further, the shape of (a) of FIG. 14 may be changed as shown in (c) of FIG. 14 upon contact with the body fluid. It may be seen that the needle body shown in (c) of FIG. 14 has a relatively decreased base diameter D0′″, but an increased needle height H′″ compared to the shape of (a) of FIG. 14. In this case, the needle diameter N′″ may be approximately the same or smaller.

Even in the needle body according to (b) and (c) of FIG. 14, any one of the base diameter D, the needle height H, and the needle diameter N as described above increases and the needle body expands upon contact with the body fluid, so that the drug contained in the needle body may be introduced into the human body.

In summary, at least one of the needle support and the needle body of the present invention may comprise the swellable polymeric material or the drug. In this regard, the needle support and the needle body may be understood as a single “microneedle”. Such a microneedle refers to a configuration of which at least a portion is inserted into an inside of the skin to introduce the drug.

For example, if the needle support and the needle body are formed by the same viscous composition, each of them is formed by the manufacturing process described above. Then, the needle support and the needle body may form a single integrated microneedle.

Although a description has been made above with reference to a preferred embodiment of the present invention, those skilled in the art will be able to make various modifications and changes to the present invention without departing from the conception and scope of the invention as described in the following claims. Therefore, all modified embodiments should be considered to be included within the technical scope of the present invention if they essentially include the elements of the claims of the present invention.

1. A microneedle patch comprising:
an adhesive sheet configured to adhere to skin;
a needle support disposed on the adhesive sheet; and
a needle body disposed on the needle support and having a cutting edge,
wherein at least one of the needle support and the needle body comprises a swellable polymeric material that is configured to expand upon contact with body fluid.

2. The microneedle patch of claim 1, wherein at least one of the needle support and the needle body comprises a drug, and
wherein at least one of the needle support and the needle body is configured to expand and deliver the drug, upon contact with the body fluid.

3. The microneedle patch of claim 1, wherein at least one of the needle support and the needle body is formed by solidifying a viscous composition in which the swellable polymeric material and a drug are dissolved.

4. The microneedle patch of claim 1, wherein the needle body comprises the swellable polymeric material and is configured in a shape having a base diameter D corresponding to a diameter of a bottom surface abutting the needle support, a needle height H corresponding to an orthogonal distance from the bottom surface to a top end, and a needle diameter N corresponding to a diameter of the cutting edge, and
wherein at least one of the base diameter, the needle height, and the needle diameter of the needle body increases upon contact with the body fluid.

5. The microneedle patch of claim 4, wherein the base diameter and the needle diameter increase by 1.1 times or greater and less than 2 times, upon contact with the body fluid.

6. The microneedle patch of claim 1, wherein the needle body comprises the swellable polymeric material and is configured in a shape having a base diameter D corresponding to a diameter of a bottom surface abutting the needle support, a needle height H corresponding to an orthogonal distance from the bottom surface to a top end, and a needle diameter N corresponding to a diameter of the cutting edge, and
wherein any one of the base diameter, the needle height, and the needle diameter of the needle body increases and any one of others decreases, upon contact with the body fluid.

7. The microneedle patch of claim 1, wherein the adhesive sheet and the needle support form a single adhesive surface, and
wherein the needle support is disposed by being inserted inwardly into the adhesive sheet from the adhesive surface, and the needle body is formed by extending outwardly from the adhesive surface.

8. The microneedle patch of claim 7, wherein the needle supports are disposed as a plurality of engraved patterns inserted inwardly into the adhesive sheet and spaced apart from one another, and
wherein the needle bodies are disposed on the needle supports, respectively.

9. The microneedle patch of claim 7, wherein the needle support is disposed as a layer inserted inwardly into the adhesive sheet and extending parallel, and
wherein the needle bodies are disposed to be spaced apart from one another on the needle support.

10. A method of manufacturing a microneedle patch, comprising:
forming a needle support on a base resin;
forming an adhesive sheet on the base resin to cover the needle support;
forming a cover resin on an upper portion of the adhesive sheet and inverting the needle support and the adhesive sheet such that the base resin is disposed above the needle support and the adhesive sheet;
removing the base resin; and
forming a needle body on the needle support,
wherein at least one of the needle support and the needle body comprises a swellable polymeric material that is configured to expand upon contact with body fluid.

11. The method of claim 10, wherein at least one of the needle support and the needle body is formed by solidifying a viscous composition in which the swellable polymeric material and a drug are dissolved.

12. The method of claim 10, wherein the forming of the needle body includes:
providing a pair of structures in each of which a base needle is formed on the needle support;
relatively moving the pair of structures closer to each other so that a pair of base needles formed at the pair of structures respectively, contact each other;
relatively moving the pair of structures away from each other so that the pair of base needles are elongated while adhering to each other and are deformed; and
forming the needle body by the deformed base needle.

13. The method of claim 10, wherein the needle support includes a plurality of supports spaced apart and formed on the base resin,
wherein the adhesive sheet is configured to cover each of the plurality of supports, and
wherein the needle body is formed on each of the plurality of supports.

14. The method of claim 10, wherein the needle bodies are formed by being disposed to be spaced apart from one another on the needle support.
